(19) United States
(12) Patent Application Publication
(10) Pub. No.: US 2023/0250520 A1
(43) Pub. Date: Aug. 10, 2023

(54) COMPOSITE MATERIAL, PROCESS FOR PRODUCING A COMPOSITE MATERIAL AND MEDICAL DEVICE BASED ON COMPOSITE MATERIAL

(71) Applicant: W&M GMBH, Graz (AT)

(72) Inventors: Bernhard WEINBERG, Graz (AT); Heinz MOITZI, Zeltweg (AT); Oliver RENK, Leoben (AT); Reinhard PIPPAN, Leoben (AT)

(21) Appl. No.: 18/007,733

(22) PCT Filed: Jun. 24, 2021
(86) PCT No.: PCT/EP2021/067283

§ 371 Date: Dec. 1, 2022

(30) Foreign Application Priority Data
Jul. 3, 2020 (AT) A60212/2020

(51) Int. Cl. C22C 38/00; C22C 33/02; C22C 23/00; C22C 18/00; C22C 1/04; B22F 3/105; A61L 31/12

(52) U.S. Cl. CPC C22C 38/002; C22C 33/0257; C22C 23/00; C22C 18/00; C22C 1/0483; C22C 1/0408; B22F 3/105; A61L 31/124; B22F 2003/1051; B22F 2202/13; B22F 2301/058; B22F 2301/30; B22F 2301/35; B22F 2999/00; B22F 2009/041

(57) ABSTRACT

The present disclosure relates to a composite material, in particular a composite material of metals, a process for producing a composite material, and a medical device, in particular an implant, based on the composite material. The composite material comprises at least 5 vol-% of Fe and at least 1 vol-% of Mg or Zn, wherein the composite material comprises a Mg or Zn phase and an Fe phase, wherein the average size of the Mg or Zn phase in at least one dimension is less than 20 μm, in particular less than 10 μm. The medical device, in particular an implant, may be suitable for fixing of bone fractures (as well as fractions of a tendon or a ligament, etc.) and/or corrections and may be capable of exhibiting a targeted failure representing a complete paradigm shift in the treatment of bone fractures and the like.

This application is the U.S. national phase of International Application No. PCT/EP2021/067283 filed 24 Jun. 2021 which designated the U.S. and claims priority to Austrian Patent Application No. A 60212/2020 filed 3 Jul. 2020, the entire contents of each of which are hereby incorporated herein by reference.

### FIELD OF THE DISCLOSURE

Embodiments of the present disclosure relate to a composite material, in particular a composite material of metals, a process for producing a composite material, and a medical device based on the composite material. The medical device, in particular an implant, may be suitable for fixing of bone fractures (as well as fractions of a tendon or a ligament, etc.) and/or corrections and may be capable of exhibiting a targeted failure representing a complete paradigm shift in the treatment of bone fractures and the like.

### BACKGROUND

Current technologies used for load-bearing orthopedic implants (typically based on titanium and its alloys or stainless steels) ignore the patient's needs for an ideal bone recovery and stick to the conventional concept of stable permanent fracture fixation. This necessitates removal surgeries to avoid osteopenia and re-fractures originating from stress shielding (due to large modulus mismatch), implant-associated infections or accumulation of nano-debris over time, which may weaken tissue or cause chronic inflammation (K. T. Kim et al., Int. J Implant Dent. 5 (2019) 10; A. Mombelli et al., Clin. Oral Implants Res. 29 (2018) 37-53).

An ideal implant solution would be (i) initially load-bearing to enable early patient mobility; (ii) dynamically self-adapting its properties (to those of the healing bone) until a tailorable and targeted preset failure occurs, which is necessary for complete bone recovery; (iii) biodegradable (so that no removal surgery is required and no waste is generated); (iv) harmless to the body (both locally and systemically, short- and long-term),

This vital combination of requirements is not comprehensively reflected in currently used permanent implants or concepts for degradable ones (such as based on magnesium (Mg), zinc (Zn) or polymers). While degradable implants cover at least some of these aspects, their application for load-bearing situations is so far limited: Implants based on pure Mg, Zn or polymers are too soft, While Mg can be strengthened by alloying with rare earth elements (REE), their use is risky: REE are non-physiological or possibly toxic, non-biodegradable and were reported to accumulate in bone and organs with thus far unknown consequences (A. Drynda et al., J. Biomed. Mater, Res. A 91A (2009) 360-369; A. Myrissa et al., Acta Biomater. 48 (2017) 521-529). Furthermore, Mg- or Zn-based materials typically degrade too fast, resulting in tissue irritation triggered by the fast release of corrosion products, particularly that of hydrogen gas (H₂), and an unwanted rapid loss of strength. Polymers degrade slowly but can induce foreign body reactions (O. M. Böstman, Pihlajamäki, Clin. Orthop. Relat. Res. 371 (2000) 216-227), Other degradable metals like iron (Fe) and its alloys provide suitable mechanical strength but degrade too slowly thereby causing the same problems as permanent implants (T, Kraus et al., Acta Biomater. 10 (2014) 3346-3353). Hence, while Mg, Zn or Fe alloys are considered attractive candidates for temporary orthopedic implants, they are rarely used in clinical practice.

In fact, a combination of Mg or Zn and Fe alloys is hindered by various physical and technological limitations: A macroscopic combination of Mg or Zn and Fe is not feasible because of a large difference of their electrochemical potentials. The formation of a macroscopic composite would create some sort of “battery”, wherein Mg or Zn would act as a sacrificial anode and consequently this would tremendously enhance the degradation rates to levels not acceptable. However, the present inventors discovered that extremely rapid electrochemical corrosion can be prevented if a Mg—Fe or Zn—Fe composite material is realized at the microscopic or even submicroscopic scale. The formation of a microscopic or sub-microscopic composite, however, is challenging in view of their enormous property difference (for instance, since Mg and Zn have a lower boiling point than the melting point of Fe, Mg and Zn boil before Fe melts; Mg and Fe are substantially immiscible and exhibit a different deformation behavior).

in light of the foregoing, there is still room for improvements of a medical device approaching an ideal implant as elucidated above.

Thus, there may be a need to provide a medical device, such as an implant suitable for fixing of bone fractures (as well as fractions of a tendon or a ligament, etc.) and/or corrections, which has initially a sufficient strength and stiffness for being load-bearing during the healing period, is dynamically self-adapting its properties (strength and stiffness to those of the healing bone) until a tailorable and targeted preset failure occurs, is biodegradable/absorbable (so that no removal surgery is required), and is harmless to the body or may even exhibit advantageous physiological effects. There may be also a need to provide a composite material, on the basis of which such a medical device may be formed.

### SUMMARY OF THE DISCLOSURE

The present inventors have made diligent studies for solving these objects and have found that a specific fine-structured composite material based on Fe and Mg or Zn may provide for a sufficient strength and stiffness to an implant capable of being load-bearing during the healing period. When implanted, the strength and stiffness of the composite material gradually reduces, thereby promoting the process of bone regrowth and as the bone has to carry more and more load, it can grow into an ideal shape so as to fulfill its natural load-bearing capability. The gradual loss in strength and in particular stiffness leads to a material exhibiting an increasing elasticity after the healing period and may ultimately result in a targeted implant failure (in particular a targeted fatigue failure of the implant) which represents a complete paradigm shift in the treatment of bone fractures and the like, Without wishing to be bound to any theory, the present inventors assume that due the specific fine structure of the composite material comprising a Mg or Zn phase and an Fe phase, wherein the average size of the Mg or Zn phase in at least one dimension (preferably at least two dimensions or all three dimensions) is less than 20 μm, in particular less than 10 μm, the anodic dissolution of Mg or Zn is slowed down for geometric reasons in that the reaction products evolving upon degradation of the Mg or Zn cannot be removed fast out of the cave that is formed during the corrosion process so that this removal will become the rate limiting process step. This means that due to the micron or nanoscaled size of the formed electrochemical element (at least in one dimension) the fluid flow through the medical device will be extremely limited for reasons of current laws. It could be confirmed by both experiment and fluid dynamics that already a reduction of the size of the electrochemical elements in one dimension is sufficient to drastically reduce the removal rates of the reaction products. As a result, the release of Mg or Zn can be controlled and tailored by appropriately adjusting the fine structure of the metallic phases of the composite material and with gradually diminishing Mg or Zn content, the remaining Fe-enriched medical device becomes more and more flexible and may ultimately result in a fatigue failure of the implant. Moreover, due to the continuous degradation of the Mg during the healing period of the bone, the thus created magnesium ions (which are osteoinductive) may stimulate and further promote the healing of the bone.

It cannot be emphasized enough that a targeted implant failure (in particular a targeted fatigue failure of the implant) as it may be achieved according to the present disclosure, represents a complete paradigm shift in the treatment of bone fractures and the like. Such targeted failure not only deviates from (or even contradicts to) the hitherto conventional approach of an enduring high load-bearing capability of an implant used in the treatment of bone fractures and the like, but can furthermore hardly be realized by the currently available bioresorbable materials based on for instance Fe or Mg or Zn or their alloys. In contrast to known bioresorbable materials, a composite material according to the present disclosure allows for appropriately adapting a degradation rate over a very broad range (several orders of magnitude) by a respective arrangement of the phases (and further of the composition, such as the Fe: Mg or the Fe:Zn ratio) so that an intentional failure of an implant can be targeted, as desired and appropriate to the individual case.

Accordingly, an exemplary embodiment relates to a composite material, in particular a composite material of metals, comprising at least 5 vol-% of Fe and at least 1 vol-% of Mg or Zn, wherein the composite material comprises a Mg or Zn phase and an Fe phase, wherein the average size of the Mg or Zn phase in at least one dimension (preferably at least two dimensions or all three dimensions) is less than 20 μm, in particular less than 10 μm (such as from 10 nm to 10 μm).

Another exemplary embodiment relates to a process for producing a composite material comprising subjecting a mixture (or blend) of an Fe-containing powder and a Mg- or Zn-containing powder to a severe plastic deformation (SPD) process.

Still another exemplary embodiment relates to a medical device (in particular an implant) based on or comprising a composite material as described herein.

Other objects and many of the attendant advantages of embodiments of the present disclosure will be readily appreciated and become better understood by reference to the following detailed description of embodiments and the accompanying drawings.

### BRIEF DESCRIPTION OF THE DRAWINGS

FIG. 1 schematically illustrates a healing process of a bone fracture following the concept of a dynamic osteosynthesis by means of an implant: according to an exemplary embodiment of the present disclosure.

FIG. 2 shows representative composite microstructures of a conceptual high pressure torsion (HPT) test on Mg50% Fe subjected to a) low strains and b) enormous strains and the corresponding hydrogen (H₂) evolution upon degradation.

### DETAILED DESCRIPTION OF EXEMPLARY EMBODIMENTS

Hereinafter, details of the present disclosure and other features and advantages thereof will be described. However, the present disclosure is not limited to the following specific descriptions, but they are rather for illustrative purposes only.

It should be noted that features described in connection with one exemplary embodiment or exemplary aspect may be combined with any other exemplary embodiment or exemplary aspect, in particular features described with any exemplary embodiment of a composite material may be combined with any exemplary embodiment of a process for producing a composite material and with any exemplary embodiment of a medical device and vice versa, unless specifically stated otherwise.

Where an indefinite or definite article is used when referring to a singular term, such as “a”, “an” or “the”, a plural of that term is also included and vice versa, unless specifically stated otherwise, whereas the word “one” or the number “1”, as used herein, typically means “just one” or “exactly one”.

The expression “comprising”, as used herein, includes not only the meaning of “comprising”, “including” or “containing”, but may also encompass “consisting essentially of” and “consisting of”.

In a first aspect, an exemplary embodiment relates to a composite material, in particular a composite material of metals, comprising at least 5 vol-% of Fe and at least 1 vol-% of Mg or Zn.

The term “composite material”, as used herein, may in particular mean a material made from two or more constituent materials with different physical or chemical properties that, when combined in a composite material, produce a material with characteristics different from the individual components. The individual components remain separate and distinct within the finished structure in contrast to alloys or solid solutions. In particular, the composite material may contain separate phases, more specifically an Mg or Zn phase and an Fe phase, as will be explained in further detail below,

The composite material comprises at least 5 vol-% of Fe and at least 1 vol-% of Mg or Zn. in an embodiment, the composite material comprises at least 10 vol-% of Fe, in particular at least 20 vol-% of Fe, in particular at least 30 vol-% of Fe, in particular at least 40 vol-% of Fe, in particular at least 50 vol-% of Fe, in particular at least 60 vol-% of Fe, and up to 99 vol-% of Fe, in particular up to 95 vol-% of Fe, in particular up to 90 vol-% of Fe, in particular up to 80 vol-% of Fe.

In an embodiment, the composite material comprises at least 5 vol-% of Mg or Zn, at least 10 vol-% of Mg or Zn, in particular at least 20 vol-% of Mg or Zn, in particular at least 30 vol-% of Mg or Zn, in particular at least 40 vol-% of

Mg or Zn, in particular at least 50 vol-% of Mg or Zn, in particular at least 60 vol-% of Mg or Zn, and up to 95 vol-% of Mg or Zn, in particular up to 90 vol-% of Mg or Zn, in particular up to 80 vol-% of Mg or Zn.

In an embodiment, a total amount of Fe and Mg or Zn is at least 80 vol-%, in particular at least 90 vol.-%, in particular at least 95 vol.-%, in particular at least 99 vol.-%.

In an embodiment, a volume ratio of Fe to Mg or Zn is from 20:80 to 80:20 (20%:80% to 80%:20%), in particular from 30:70 to 70:30, in particular from 40:60 to 60:40. By taking this measure, particular suitable material characteristics may be achieved.

In an embodiment, in addition to the essential components Mg or Zn and Fe, the composite material may further comprise at least one element selected from the group consisting of Ca (for instance up to 5 wt-%, in particular up to 2 wt-%), Mn (for instance up to 30 wt-%, in particular up to 20 wt-%), Zr (for instance up to 2 wt-%), Y (for instance up to 5 wt-%), Nd (for instance up to 5 wt-%), Gd (for instance up to 10 wt-%), Sn (for instance up to 5 wt-%) and Al (for instance up to 5 wt-%). Moreover, in case of an Fe-Mg composite material, the composite material may further comprise Zn (for instance up to 5 wt-%, in particular up to 2 wt-%). In case of an Fe—Zn composite material, the composite material may further comprise Mg (for instance up to 5 wt-%, in particular up to 2 wt-%). Mg, Ca and Zn may in particular be comprised when using an Mg—Ca—Zn alloy as an Mg source for producing the composite material and may consequently in particular be present in the Mg phase of the composite material. Mn may in particular be comprised when using a nonmagnetic Fe—Mn alloy as an Fe source for producing the composite material and may consequently in particular be present in the Fe phase of the composite material. In addition, the composite material may further comprise inevitable impurities, in particular in a total amount of up to 1 wt-%, preferably up to 0.5 wt-%, more preferably up to 0.1 wt-%, and/or wherein each impurity is contained in an amount of not more than 0.1 wt-%, preferably not more than 0.05 wt-%, more preferably not more than 0.01 wt-%.

The composite material comprises a Mg or Zn phase and an Fe phase. In particular, the composite material may comprise a crystalline Mg or Zn phase and a crystalline Fe phase The term “Mg or Zn phase”, as used herein, may in particular denote a distinct portion of the composite material wherein the magnesium or zinc of the composite material is predominantly present, in particular in crystalline form. For instance, more than 80%, in particular more than 90% and even substantially all of the magnesium or zinc of the composite material may be present in the Mg or Zn phase, The Mg or Zn phase may thus also be referred to as a Mg- or Zn-enriched or Mg- or Zn-rich phase. Likewise, the term “Fe phase”, as used herein, may in particular denote a distinct portion of the composite material wherein the iron of the composite material is predominantly present, in particular in crystalline form. For instance, more than 80%, in particular more than 90% and even substantially all of the iron of the composite material may be present in the Fe phase, The Fe phase may thus also be referred to as an Fe-enriched or Fe-rich phase. As will be readily understood by a person skilled in the art, the composite material may comprise a plurality of Mg or Zn phases and Fe phases.

In an embodiment, a volume ratio of the Fe phase to the Mg or Zn phase is from 20:80 to 80:20 (20%:80% to 80%:20%), in particular from 30:70 to 70:30, in particular from 40:60 to 60:40.

The composite material may in particular be characterized in that the average size of the Mg or Zn phase in at least one dimension (preferably at least two dimensions or even all three dimensions) is less than 20 μm, in particular less than 10 μm (such as from 10 nm to 10 μm). It has turned out that already a reduction of the size of the Mg or Zn phase in one dimension is sufficient to drastically reduce the degradation rates of Mg or Zn. The present inventors assume that due to the specific fine structure of the composite material comprising a Mg or Zn phase and an Fe phase, wherein the average size of the Mg or Zn phase in at least one dimension is less than 20 μm, in particular less than 10 μm, the anodic dissolution of Mg or Zn is slowed down for geometric reasons in that the reaction products evolving upon degradation of the Mg or Zn cannot be removed fast out of the cave that is formed during the corrosion process so that this removal will become the rate limiting process step. Thus, the release of Mg or Zn can be controlled and tailored by appropriately adjusting the fine structure of the metallic phases of the composite material and with diminishing Mg or Zn content, the remaining Fe-enriched medical device becomes more and more flexible (elastic) and may ultimately result in an implant failure (in particular a fatigue failure of the implant).

The term “average size of the Mg or Zn phase”, as used herein, may in particular denote an average value (in particular an arithmetic mean) of the sizes of a plurality of Mg or Zn phases in the composite material. The term “at least one dimension”, as used herein, may in particular mean at least one of the three spatial dimensions (length, width and height) of a three-dimensional Mg or Zn phase (i.e. a distinct portion of the composite material wherein the magnesium or zinc of the composite material is predominantly present). Accordingly, the term “at least two dimensions”, as used herein, may in particular mean at least two of the three spatial dimensions (length, width and height) of a three-dimensional Mg or Zn phase. in an embodiment, the average size of the Mg or Zn phase in at least one dimension (preferably at least two dimensions or all three dimensions) is less than 5 μm (such as from 200 nm to 5 μm), in particular less than 3 μm (such as from 500 nm to 3 μm). The average size of the Mg or Zn phase in at least one dimension may in particular be less than 10 μm, in particular less than 5 μm, in particular less than 3 μm and may in particular be at least 10 nm, in particular at least 25 nm, in particular at least 50 nm, in particular at least 100 nm, in particular at least 200 nm, in particular at least 500 nm. By appropriately adjusting the fine structure of the metallic phases of the composite material, the release of Mg or Zn may be tailored as desired.

The average size of the Mg or Zn phase may be determined for instance by visual microscopic observation with an appropriate magnification, for instance by using a light optical microscope, an electron microscope (such as a transmission electron microscope (TEM) or a high resolution scanning electron microscope (SEM)) and by randomly selecting an appropriate number of Mg or Zn phases and calculating the average (such as an arithmetic mean) of the individual sizes.

In an embodiment, the average grain size within the Fe phase and/or within the Mg or Zn phase is less than 5 μm, in particular less than 1 μm, In particular, it may be advantageous if the average grain size in the Fe phase is less than 5 μm, in particular less than 1 μm. By taking this measure, the strength of the composite material may be significantly raised and may consequently enable a high load bearing capability of a medical device, in particular an implant, based thereon.

Similar to the determination of the average size of the Mg or Zn phase, the determination of an average grain size is known to a person skilled in the art and can be performed for instance by visual microscopic observation with an appropriate magnification, for instance by using a light optical microscope, an electron microscope (such as a transmission electron microscope (TEM) or a high resolution scanning electron microscope (SEM)) and by randomly selecting an appropriate number of grains and calculating the average of the individual grain sizes.

In an embodiment, the composite material is obtainable (or obtained) by a severe plastic deformation (SPD) process. The term “severe plastic deformation”, as used herein, may in particular refer to a group of metal forming techniques which allow to apply enormous strains, Typically, they involve a complex stress state or high shear strains which generate a huge defect density within the material resulting in significant grain refinement well below a micron. SPD processes further offer the possibility to consolidate fine metallic powders into bulk composites. SPD processes, a combination of different SPD processes or a combination of spark plasma sintering (SPS) or comparable processes which allow to pre-consolidate metallic powders into bulk pieces and their subsequent deformation by SPD processes or conventional metal forming processes that allow for large applied strains (e.g. rolling, wire drawing or swaging) may be suitable for preparing a composite material as described herein.

In an embodiment, the severe plastic deformation process comprises a high pressure torsion (HPT) process. The term “high pressure torsion”, as used herein, may in particular denote a SPD process wherein a sample is subjected to enormous hydrostatic pressures and subsequently large shear strains are applied. In case of powder precursors this ensures densification into a bulk material or composite. More specifically, powder blends may be consolidated and refined to dense and bulk (nano)composites. A HPT process has proven to be particularly appropriate to vary the structural size of the Mg or Zn and Fe phase as well as their geometric arrangement in a manifold manner. In addition, HPT may allow for an easier adjustment of composite architecture compared to e.g. spark plasma sintering in combination with multi forging or drawing.

In an embodiment, the composite material has a yield strength of more than 200 MPa, in particular of more than 300 MPa. As a result, a medical device, in particular an implant, based on the composite material may exhibit a sufficient strength for being load-bearing during the healing period when used for fixing bone fractures. The yield strength may in particular be determined in accordance with DIN EN ISO 6892-1 (tensile testing) or DIN 50106:2016-11 (compression testing).

In an embodiment, the composite material has a Young's modulus of less than 200 GPa, in particular of less than 150 GPa. As a result, a medical device, in particular an implant, based on the composite material may exhibit a sufficient stiffness for being load-bearing during the healing period. The Young's modulus may in particular be determined in accordance with DIN EN ISO 6892-1 (tensile testing) or DIN 50106:2016-11 (compression testing). Alternatively, the Young's modulus may also be determined in accordance with DIN EN ISO 14577-1:2015-11.

In a second aspect, another exemplary embodiment relates to a process for producing a composite material (in particular a composite material according to the first aspect) comprising subjecting a mixture (or blend) of an Fe-containing powder and a Mg- or Zn-containing powder to a severe plastic deformation process.

In an embodiment, the Fe-containing powder comprises pure Fe or an Fe alloy, such as a (non-ferromagnetic) Fe Mn alloy.

In an embodiment, the Mg-containing powder comprises pure Mg or an Mg alloy, such as a Mg—Ca—Zn alloy, a Mg—Zr alloy, a Mg—Y alloy, a Mg—Gd alloy, a Mg—Sn alloy, a Mg—Al alloy and combinations of these alloying elements.

In an embodiment, the Zn-containing powder comprises pure Zn or an Zn alloy, such as a Zn—Mg alloy, a Zn—Al alloy, a Zn—Ca alloy, a Zn—Sn alloy and combinations of these alloying elements.

In an embodiment, the Fe-containing powder and/or the Mg- or Zn-containing powder have an average particle size of less than 40 μm, in particular less than 20 μm. The smaller the average particle size of the raw material, the less efforts may be required in subsequent severe plastic deformation. Moreover, by taking this measure, a reduced degradation rate of the composite material can be obtained at a given phase fraction of Mg or Zn and Fe and the same applied strain.

In an embodiment, the severe plastic deformation process comprises a high pressure torsion process. Further details for both the severe plastic deformation (SPD) as well as high pressure torsion (HPT) process have been given above in the context of the first aspect.

In an embodiment, the severe plastic deformation process, in particular the high pressure torsion, is carried out under a pressure of at least 2 GPa, in particular at least 3 GPa, and up to 15 GPa, in particular up to 12 GPa.

In an embodiment, the severe plastic deformation process, in particular the high pressure torsion, is carried out at an elevated temperature, for instance at a temperature of at least 200° C., in particular at least 300° C. By taking this measure, a reduced degradation rate of the composite material can be obtained at a given phase fraction of Mg or Zn and Fe and the same applied strain.

in a third aspect, still another exemplary embodiment relates to a medical device based on or comprising a composite material as described herein. In particular, the medical device may be an implant.

The term “implant”, as used herein, may in particular mean a medical device that may be at least partially implanted in a human or animal body. The implant may have various two- or three-dimensional shapes, such as a pin, a rod, a screw, a wire, a plate, a disc, a dome or a hemisphere. In particular, the implant may be selected from the group consisting of a plate, a nail, a screw, and a surgical staple; i.e. the implant may be shaped as a plate, a nail, a screw and/or a surgical staple. The implant may in particular be at least partially resorbable (biodegradable) by a human or animal body once implanted. The implant may in particular be configured for a fixation of bone, tendon or ligament fractures and/or corrections, in particular for an internal fixation of bone, tendon or ligament fractures and/or corrections.

The expression “medical device/implant based on a composite material”, as used herein, may in particular mean that the medical device or implant comprises a composite material, in particular as an essential constitutional component thereof. It may even mean that the implant (substantially) consists of such composite material or is made of such composite material.

In an embodiment, the medical device, such as the implant, has a yield strength of more than 200 MPa and/or a Young's modulus of less than 200 GPa. In this embodiment, the medical device may be preferably configured such that the value of at least one of the yield strength and/or the Young's modulus is less than 50% of the respective initial value after the threefold (three times) healing period, in particular after the twofold (twice, double) healing period, To this end, the release of Mg or Zn may be tailored by appropriately adjusting the fine structure of the metallic phases of the composite material and with gradually diminishing Mg or Zn content, the strength and stiffness of the medical device may decrease, i.e. the yield strength and the Young's modulus of the material constituting the medical device may decrease.

The term “healing period”, as used herein, may in particular mean a meaningful duration of a healing process, in particular for healing of fractions of a bone, a tendon or a ligament, and may thus differ accordingly.

In an embodiment, the medical device, in particular the implant, is configured such that failure (such as breakage) of the medical device occurs after the healing period, but preferably before the threefold (three times) healing period. To this end, the release of Mg or Zn may be tailored by appropriately adjusting the fine structure of the metallic phases of the composite material and with diminishing Mg or Zn content, the remaining Fe-enriched medical device becomes more and more flexible and may ultimately result in a fatigue failure of the implant.

The present disclosure is further described by reference to the accompanying figures, which are solely for the purpose of illustrating specific embodiments and shall not be construed as limiting the scope of the disclosure in any way.

Before, referring to the drawings, exemplary embodiments will be described in further detail, some basic considerations will be summarized based on which exemplary embodiments of the disclosure have been developed.

To date load bearing and absorbable implants remain their integrity much longer than required. Due to the required high strength during the first four weeks, the degradation rates are tuned to low levels and consequently, the supportive function for the bone exists much longer than useful for an optimal bone regrowth.

The implementation of a self-adapting implant with loosing stiffness and desired failure according to an embodiment of the disclosure is deliberately designed to stimulate bone regrowth in an optimal way and is thus radically new and innovative.

The load transfer from the implant to the bone and the desired failure can be tailored by a sufficient reduction of strength and elastic moduli over time. While a load transfer from the medical device (MD) to the bone is self-explaining if the strength of the material is reduced, there is also evidence that a reduction of the elastic modulus of the implant can be used for the desired load transfer and thus optimized healing conditions. It becomes plausible considering a support structure having a 10 times reduced stiffness compared to the object that needs to be supported. In such a case, there will be no support at all and the object itself will carry the load. Exactly the same effect would occur in case of fixations used for fractured bones, however already for much smaller stiffness reductions. Consequently, the load bearing function of the bone is stimulated continuously towards its natural condition.

The proposed function of the absorbable implant according to an embodiment of the disclosure can accordingly be divided into three parts:

Part 1: Supporting and load bearing function with material parameters (strength, stiffness) at its maximum values or at least much larger than the values of human bone for T<T1.

Part 2: Reduction of these values between T1 and T2 to an adjustable and stimulating level optimal for bone regrowth, with an eventual failure if desired.

Part 3: Continuation of the usual and known absorption process for the time period after T2 (i.e. this final absorption process of the residuals can take eventually up to 1-3 years).

It should be noted that T1 is larger than the healing period, but T2 is shorter than the threefold healing period. In the ideal case, T2 is shorter than twice or 1.5 times the healing period.

To reach this goal, several absorbable and biocompatible materials have been investigated. It has been observed that organic materials are generally suitable to adjust the degradation rates properly. Nevertheless, due to their strength levels and their initial degradation period a versatile usage with a single material is not possible at all.

Considering metals, predictions of a desired failure of the implant are extremely difficult for magnesium, zinc and their alloys. This is because of the initially high hydrogen (H₂) gas evolution and on the other hand because the degradation rate strongly depends on the metabolism which can vary from patient to patient. Iron on the other hand can rarely be used, as it degrades too slowly.

In principle one could consider a composite of Mg or Zn and Fe which would unite the advantages of both materials. However, a macroscopic combination of Mg or Zn and Fe is not feasible because of their large difference of the electrochemical potential. Formation of a macroscopic composite would create a ‘battery’, in which Mg or Zn would act as a sacrificial anode and consequently this would tremendously enhance the degradation rates to levels not acceptable. Accordingly, a Mg—Fe or Zn—Fe composite needs to be realized on a microscopic or even sub-microscopic scale to prevent an extremely fast electrochemical corrosion, as mentioned.

Due to the extremely different melting points of Mg or Zn and Fe, composites of Mg or Zn and Fe cannot be synthesized by conventional processes. For this reason, other processing techniques need to be targeted and the HPT process offers a perfect solution to fabricate such composites.

The HPT process does not only allow to generate a composite of Mg or Zn and Fe, but additionally ensures that the geometric arrangement and the size of the two phases can be varied in a wide range by a change of the process parameters. A change of the phase spacing and their geometric arrangement consequently allow to adjust the degradation rates and the strength of the composites in a wide range.

Furthermore, simple approaches (e.g. regions with different structural sizes and/or diameters, etc.) additionally allow to adjust (accelerate or retard) the degradation rates and so the initial hydrogen gas (H₂) evolution or allow for a faster failure after the initial healing period,

The proposed combination of Mg or Zn and Fe into a composite for MD offers a whole range of further possibilities:

1) SPD processes, a combination of different SPD processes or a combination of spark plasma sintering (SPS) or comparable processes which allow to pre-consolidate metallic powders into bulk pieces and their subsequent deformation by SPD processes or conventional metal forming processes that allow for large applied strains (e.g. rolling, wire drawing or swaging) offer the synthesis of Mg or Zn and Fe powders into a fully dense bulk compound. Especially the HPT process is appropriate to vary the structural size of the Mg or Zn and Fe phase as well as their geometric arrangement manifold.

2) If the two constituents Mg or Zn and Fe are perfectly bonded (possible for instance by the HPT process), the elastic moduli of the compound are high, with values between that of pure Mg or pure Zn and pure Fe. If during degradation of the composite Mg or Zn is preferentially dissolved, the stiffness of the remaining Fe skeleton drops significantly. The load bearing capability (strength) still remains on a rather high level as Fe has a much higher strength than Mg or Zn.

3) If the phase spacing of Mg or Zn and Fe are small, the anodic dissolution of Mg or Zn is slowed down for geometric reasons. The reaction products evolving upon degradation of the Mg or Zn cannot be removed fast out of the cave that forms during the corrosion process and this removal will become the rate limiting process. This means that due to the micron or nanoscaled size of the formed electrochemical element (at least in one dimension) the fluid flow through the MD will be extremely limited for reasons of current laws. Both, experiment and fluid dynamics confirm that already a reduction of the size of the electrochemical elements in one dimension is sufficient to massively reduce the removal rates of the reaction products.

With the solution proposed by the disclosure the degradation of the Mg or Zn phase of the MD can be retarded by 20%, preferentially by 50% compared to a macroscopic but identical electrochemical system.

As the spacing of the Mg or Zn at least in one dimension is well-below 20 μm, a massive reduction of the degradation rate is achieved. Additional experiments have proven that Mg or Zn spacing of 50 nm to 10 μm, preferentially 200 nm to 5 μm, ideally 500 nm to 3 μm are perfectly suited for application in a MD. It should be noted again that these dimensions are required only along one of the principle axis while along other directions the dimensions of the Mg or Zn phase can still be from several microns up to a millimeter.

4) If Mg or Zn and Fe form a bulk and fully dense composite, right after implantation the elastic moduli are rather high. Due to the corrosion of the Mg or Zn, however, the remaining Fe skeleton becomes very flexible due to its comparably low stiffness.

This peculiarity of the proposed disclosure can be preferentially applied to MDs. At the beginning the stiffness of the overall MD is higher than that of the bone structure (healthy condition) to be supported after the fracture. The MD will carry the load and the bone is protected, supported by the MD and the healing process can start.

During the healing period of the fractured bone the Mg or Zn dissolves and consequently the MD becomes less stiff and more flexible than the supported bone structure. The bone needs to carry continuously more load what is beneficial for the healing process and healthy bone structure formation.

By varying the structural size of the Mg or Zn and Fe phase as well as their architectural arrangement in the MD the degradation rate of the Mg or Zn phase can be tailored in a wide range. Altering the volume fractions of Mg or

Zn and Fe as well as the process parameters in HPT enables to change the structural scale of the constituents over four orders of magnitude (100 μ-10 nm).

Furthermore, the Mg or Zn and Fe phase can be geometrically arranged in different ways. For instance, a composite of the same composition and structural scale could consist of a layered, alternating arrangement of Mg or Zn and Fe but could as well be made up of finely dispersed and isolated Mg or Zn or Fe phase. In doing so, the flow stress and/or the stiffness of the MD after the healing period and before the threefold healing period can be reduced by 10%, preferentially by more than 20%, ideally by more than 30% compared to the initial stiffness.

5) If the Mg or Zn is dissolved, the remaining Fe skeleton becomes flexible and less stiff. This is accompanied by enhanced deformation within the Fe skeleton and the higher stress levels drastically shorten the fatigue lifetime. Fracture may thus finally occur despite the Fe degradation has not significantly started or would take more time. This allows for a concept involving a deliberately chosen failure of the Fe skeleton once bone healing is completed. A long-lasting supportive function of the MD which is not preferred from a medical viewpoint can thus be excluded.

6) Due to the continuous degradation of the Mg or Zn during the healing period of the bone, the released Mg ions are osteoinductive, what stimulates and supports the healing of the bone.

7) Due to the reduced dimensions of the Mg or Zn phase and the respective reduced degradation rate (compare 3) the problematic release of hydrogen gas bubbles can be significantly reduced, what is preferred from a medical point of view.

8) The HPT process and SPD processes in general strengthen the Mg or Zn and Fe phase. With grain sizes below 5 μm, preferentially below 1 μm, the strength of Fe is raised from typically 100 MPa to more than 1000 MPa. This enormous strengthening of the Fe phase enables a huge load bearing capability of the MD of more than 300 MPa.

9) As the degradation of the Mg or Zn phase proceeds faster than that of the Fe phase, the surface of the Fe which is in contact with body fluid is enhanced what accelerates the generally low degradation rate of Fe.

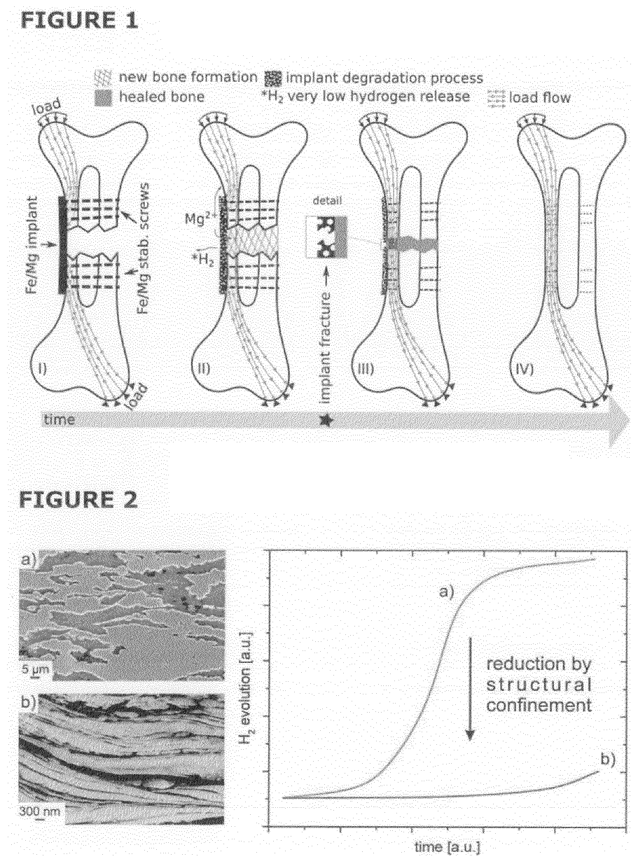

FIG. 1 schematically illustrates a healing process of a bone fracture following the concept of a dynamic osteosynthesis by means of an implant according to an exemplary embodiment of the disclosure.

FIG. 1.I) illustrates an initial stabilization of the bone fracture by means of an implant based on an Fe/Mg composite according to an embodiment of the disclosure. At this initial stage of the healing process, the implant exhibits a sufficient strength and stiffness capable of taking over the natural load-bearing function of the bone.

With proceeding healing process, the Fe/Mg composite material gradually degrades by dissolution of Mg, as shown in FIG. 1.II). As a result of this gradual dissolution, the strength and stiffness of the remaining Fe-enriched implant reduces, thereby promoting the bone regrowth (supported by an osteoinductive effect of released magnesium ions) and as the bone has to carry more and more load, it can grow into an ideal shape so as to fulfill its natural load-bearing capability.

After completion of the bone fracture healing, the implant may be degraded to such an extent that it fractures itself, as shown in FIG. 1.III), and may eventually become completely absorbed over time, as shown in FIG. 1.IV).

FIG. 2 shows representative composite microstructures of a conceptual high pressure torsion (HPT) test on Mg50% Fe subjected to a) low strains and b) enormous strains and the corresponding hydrogen (H₂) evolution upon degradation.

On the left side of FIG. 2, scanning electron microscope images of a composite material made from Mg and Fe in equal amounts subjected to a) low strains; b) enormous strains are shown. As evident from this micrographs, a particular fine-structured composite material of Fe and Mg may be obtained by applying high strains. On the right side of FIG. 2, their effect on corrosion rates is schematically shown. As evident from this graph, a significant reduction of Mg dissolution (as indicated by the generation of hydrogen gas (H₂)) can be achieved by structural confinement.

While the present disclosure has been described in detail by way of specific embodiments and examples, the disclosure is not limited thereto and various alterations and modifications are possible, without departing from the scope of the disclosure.

1. A composite material comprising
at least 5 vol-% of Fe;
at least 1 vol-% of Mg or Zn;
wherein the composite material comprises a Mg or Zn phase and an Fe phase, wherein the average size of the Mg or Zn phase in at least one dimension is less than 20 μm.

2. The composite material according to claim 1, wherein the composite material comprises at least 10 vol-% of Fe.

3. The composite material according to claim 1, wherein the composite material comprises at least 10 vol-% of Mg or Zn.

4. The composite material according to claim 1, wherein a total amount of Fe and Mg or Zn is at least 80 vol-%.

5. The composite material according to wherein a volume ratio of Fe to Mg or Zn is from 20:80 to 80:20.

6. The composite material according to claim 1, further comprising at least one element of the group consisting of Ca, Mg, Zn, Mn, Zr, Y, Nd, Gd, Sn and Al as well as inevitable impurities.

7. The composite material according to claim 1, wherein the average size of the Mg or Zn phase in at least one dimension is less than 5 μm.

8. The composite material according to claim 1, wherein the average grain size in the Fe phase and/or in the Mg or Zn phase is less than 5 μm.

9. The composite material according to claim 1, obtainable by a severe plastic deformation process.

10. The composite material according to claim 9, wherein the severe plastic deformation process comprises a high pressure torsion process.

11. The composite material according to claim 1, wherein the composite material has a yield strength of more than 200 MPa.

12. The composite material according to claim 1, wherein the composite material has a Young's modulus of less than 200 GPa.

13. A process for producing a composite material comprising
subjecting a mixture of an Fe-containing powder and an Mg- or Zn-containing powder to a severe plastic deformation process.

14. The process according to claim 13, wherein the severe plastic deformation process comprises a high pressure torsion process.

15. A medical device based on a composite material comprising:
at lest 5 vol-% of Fe,
at least 1 vol-% of Mg or Zn,
wherein the composite material comprises a Mg or Zn phase and an Fe phase wherein the average size of the Mg or Zn phase in at least one dimension is less than 20 μm.

16. The medical device according to claim 15, wherein the medical device is an implant.

17. The medical device according to claim 15, wherein the implant is selected from the group consisting of a plate, a nail and a screw.

18. The medical device according to claim 15 having a yield strength of more than 200 MPa and/or a Young's modulus of less than 200 GPa.

19. The medical device according to claim 18, wherein the medical device is configured such that the value of at least one of the yield strength and/or the Young's modulus is less than 50% of the respective initial value after the threefold healing period.

20. The medical device according to claim 15, wherein the medical is configured such that failure of the medical device occurs after the healing period.
